(19) United States
(12) Patent Application Publication
(10) Pub. No.: US 2008/0213293 A1
(43) Pub. Date: Sep. 4, 2008

(54) Prevention and Treatment of Recurrent Respiratory Papillomatosis

(75) Inventors: Kenneth E. Palmer, Owensboro, KY (US); Daniel Tuse, Vacaville, CA (US); Stephen J. Reinl, Sacramento, CA (US); Mark L. Smith, Davis, CA (US); Gregory P. Pogue, Austin, TX (US)

Correspondence Address: STITES & HARBISON, PLLC, 400 W MARKET ST, SUITE 1800, LOUISVILLE, KY, 40202-3352, US

(73) Assignee: LARGE SCALE BIOLOGY CORPORATION, Vacaville, CA (US)

(21) Appl. No.: 11/950,366

(22) Filed: Dec. 4, 2007

Related U.S. Application Data
(63) Continuation of application No. 11/107,575, filed on Apr. 15, 2005, now abandoned.
(60) Provisional application No. 60/563,071, filed on Apr. 15, 2004.

(51) Int. Cl. A61K 39/00; A61K 38/00; A61P 37/00

(52) U.S. Cl. 424/186.1; 514/2

(57) ABSTRACT

Juvenile-onset recurrent respiratory papillomatosis is treated using active vaccination or passive immune therapy of neutralizing antibodies against HPV L2 neutralizing epitopes.

### CROSS-REFERENCE TO RELATED APPLICATIONS

This application is a Continuation of U.S. application Ser. No. 11/107,575, filed Apr. 15, 2005, which is based on and claims benefit of U.S. Provisional Application No. 60/563,071, filed Apr. 15, 2004, entitled “METHOD FOR PREVENTION OF PAPILLOMAVIRUS-ASSOCIATED DISEASE IN BABIES AND CHILDREN”, both of which are incorporated herein by reference.

### BACKGROUND OF THE INVENTION

1. Field of the Invention

The present invention relates vaccines and their use to prevent and treat recurrent respiratory papillomatosis.

2. Description of Prior Art

Human papillomaviruses cause a number of different pathologies of varying severity. Of particular concern are those which cause genital warts. The most common papillomaviruses are the genital wart-associated HPV-6 and HPV-11, as well as the viruses implicated in the etiology of cervical cancer such as HPV-16, HPV-18, HPV-31; HPV-33; HPV-35 and HPV-39 (Peñaloza-Plascencia et al., 2000).

Research in the past decade has generated a wealth of knowledge on the correlates of protection against papillomavirus infection. However, the breadth of antigenic diversity present in this group of pathogens makes induction of broadly neutralizing antibodies through current modes of vaccination very difficult. People have attempted to develop vaccines to prevent infection with two (HPV-16 and HPV-18) of the fifteen “high risk” human genital papillomaviruses known to cause cervical, anogenital and other mucosal cancer. Likewise, others have proposed doing the same for other types of HPV to prevent genital warts, but this suffers from several drawbacks. The most important of these is that the vaccine compositions do not appear to induce good cross-protective immunity, so while they have high likelihood of protecting women against infection with HPV-16 and HPV-18, they are unlikely to protect against infection with other types—a finding that may present a significant barrier to FDA approval.

In recent years papillomavirus prophylactic vaccine development has focused on a single product: virus-like particles derived from the major capsid protein, and this is the composition that is in late stage clinical trials. However, recent data indicates that the minor capsid protein (L2), unlike L1, contains epitopes that can induce antibodies with neutralizing activities functional against a broad range of papillomavirus types. The first generation of HPV L2 vaccines that were tested were composed of short peptides. These were poorly immunogenic and neutralizing titers induced on vaccination have been low, and consequently the outcome of vaccination is highly variable (18, 30, 32, 33), albeit promising from the perspective that cross-neutralizing antibodies were induced. Given that epidemiologists predict that HPV vaccines should optimally protect against at least seven high risk types, there is a need for papillomavirus vaccines that are highly immunogenic and preferably composed of a single immunogen, yet capable of generating antibodies with broadly cross-neutralizing activity. The L2 protein is an attractive target antigen for solving this problem, but there are no data on the ability of larger portions of L2 to induce broadly neutralizing antibodies and the L2 protein itself is poorly immunogenic.

These viruses can also infect babies born to infected mothers. In such a situation, the child may develop papillomas in the respiratory tract which can interfere with breathing. This occurs in about 4 per 100,000 children and about 7 in 1,000 children born to mothers with vaginal condyloma.

Recurrent respiratory papillomatosis (RRP) is a seriously debilitating disease caused by infection with mucosal tissue-tropic papillomavirus types. Lesions are commonly found in the larynx and on the vocal cords, but can spread to the trachea and lungs. The respiratory papillomas caused by infection by papillomaviruses can be deadly in pediatric RRP due to the small size of the upper airway in children. Lesions may grow very fast and papillomatosis can cause overwhelming neoplasia in the respiratory tree. Death can result from airway obstruction, cancerous transformation, overwhelming spread of the disease, or complications from surgical treatments (reviewed by Shykhon et al., 2002).

The morbidity associated with juvenile onset RRP (JORRP) is extremely severe, with affected children requiring, on average, 5.1 surgeries annually (Reeves et al., 2003). RRP is also associated with significant economic burden, in excess of $445,000 lifetime cost for adult-onset RRP (International RRP ISA Center). Surgery is currently the preferred treatment. Others have used interferon-alpha2a, retinoic acid and indol-3-carbinol/diindolylmethane and cidofovir. Stressgen Inc. has proposed a therapeutic vaccine product in clinical trials based on the HPV E7 gene fused to a mycobacterial heat shock protein. The Stressgen product shows some promise for therapy of established lesions, but cannot prevent transmission of the etiological agents to patients at high risk.

Juvenile onset recurrent respiratory papillomatosis (JORRP) is caused by infection of the upper respiratory tract of infants and children with mucosal tissue-tropic papillomaviruses, particularly HPV-6 and HPV-11. It is accepted that JORRP is a perinatally-acquired infectious disease (Shah et al., 1998). It is caused by infection of newborns with mucosal tissue-tropic papillomaviruses, usually HPV-11 or HPV-6. This infection is usually transmitted to the nasopharynx of a newborn from the genital tract of its mother during vaginal delivery, although there is also evidence of transmission to the fetus in utero. Reeves et al. (2003) estimate the incidence of JORRP at 1.7 to 2.6 per 100,000 children in the USA; this corresponds to around 2300 new cases of JORRP annually. The disease is most commonly diagnosed in children between 2 and 3 years of age. In the USA, at least $110 million is spent annually on the problem.

The risk factors for JORRP disease are: (1) presence of condylomas (genital warts) in the mother; (2) first births and (3) young maternal age (Shah et al., 1998). Some gynecologists recommend Cesarean section births when a pregnant woman presents with obvious condyloma, but this does not completely avoid transmission of HPV to the neonate. In addition, the cost associated with elective Cesarean section delivery can be prohibitive; many women presenting with genital warts come from lower socio-economic sectors of society where adequate health care reimbursement is not available. Women with subclinical, undetected HPV infection may also transmit virus to their newborn during vaginal delivery.

Epidemiological studies indicate that 1% of the women of childbearing age in the USA have visible genital warts and that a further 15% percent of the population have subclinical infection (Koutsky, 1997). Silverberg et al. (2003) found that the risk of giving birth to offspring with JORRP is 231.4 times higher in women with HPV-6 or HPV-11 genital lesions. However, only 0.7% of births to women infected with genital warts results in JORRP in their children. Clearly other factors impact transmission and productive infection of the respiratory tract with perinatally-transmitted HPV. Gelder et al. (2003) found a that presence of HLA DRB1*0301 conferred significant risk for development of RRP, indicating that individuals carrying this allele suffer a defect in efficient detection of infection by CD4+ T-cells, and hence fail to clear infection. It is well known that papillomaviruses are very effectively neutralized by antibodies targeted to epitopes in both L1 and L2 structural proteins, and that people with papillomavirus infection develop virus-neutralizing antibodies. While neutralizing antibodies alone (Embers et al. 2002; Koutsky et al., 2002) confer protection against de novo infection with papillomaviruses, cell-mediated immune responses appear necessary for clearance of established virus infection in people without pre-existing antibodies.

The diversity of HPV types involved in the etiology of cervical cancer and genital warts is not widely appreciated, and presents significant hurdles for development of broadly applicable vaccines and therapeutics. Moreover, it is not widely recognized that the HPV-associated disease problem is not restricted to cervical cancer and genital warts.

Although there is no currently licensed vaccine to prevent infection with human papillomavirus, an extensive body of literature supports the concept that immunization with papillomavirus structural proteins L1 and/or L2 may prevent papillomavirus infection in animal models (reviewed by Campo, 2002), and in humans (Koutsky et al., 2002). Virus neutralizing antibodies appear to be both necessary and sufficient for protection against papillomavirus infection (Embers et al., 2002; Tobery et al., 2003). Nonetheless, papillomavirus infection in vivo continues in the presence of antibodies.

Virus-neutralizing antibodies may be induced by papillomavirus infection (Christensen et al., 2000; Kawana et al., 2003a) or by vaccination with (1) chemically-inactivated virus; (2) recombinant virus-like particles composed of papillomavirus structural proteins (L1 only, or L1 and L2); (3) proteins or peptides derived from the L2 structural proteins. The L1 antigen is generally considered the major immunodominant epitope. While L2 antigen has poor ability to induce an immune response, it is still antigenic. The first strategy—chemically inactivated virion vaccines—is impractical for human vaccination as papillomaviruses are not easily cultured in vitro and lesions yield only small amounts of virus. The second strategy—recombinant virus-like particle vaccines based on L1 proteins—generates high titers of potent virus neutralizing antibodies, but neutralizing activity is papillomavirus type specific. Thus, vaccination with HPV-16 VLPs will generate specific neutralizing antibodies but will not be useful for protection against HPV-6, or HPV-11 infection.

### SUMMARY OF THE INVENTION

The object of the present invention is to prevent and treat recurrent respiratory papillomatosis (RRP), particularly juvenile onset recurrent respiratory papillomatosis (JORRP).

It is a further object of the present invention is a method and vaccine for inducing an immune response to papillomavirus in a patient with RRP or at risk for RRP.

It is another object of the present invention is a method and vaccine to induce or boost antibodies in a mother to provide antibodies to a fetus by way of transplacental transfer.

It is yet another object of the present invention is a method and immunogen to elicit an immune response in an animal and to transfer antibodies and/or cells responsible for cellular immunity to a patient at risk for acquiring or already having RRP.

It is still another object of the present invention to prepare and transfer antibodies and or cells responsible for cellular immunity to a mother at risk of transferring HPV to a fetus, newborn or infant.

It is another further object of the present invention to prepare human milk and/or colostrum containing protective or therapeutic antibodies for administration to an infant exposed to papillomavirus.

It is yet another further object of the present invention to prevent or treat adult-onset papillomatosis.

It is a still another object of the present invention to provide a topical medicament containing HPV neutralizing antibodies or cellular factors for application to the mother or infant during delivery.

Other aspects of the invention include discovering improved vaccine compositions with broader protection. Given that a current proposed vaccine, a L1 VLP, generates powerful neutralization responses against homologous virus, but little or no protective responses against non-homologous viruses, the desire to induce or produce a broadly neutralizing antibody responses are directed to the use of L2.

The present invention of treating or preventing respiratory papillomatosis is performed by immunizing either the mother or the child before, during or after delivery or administering antibody or cellular components against HPV to prevent or treat respiratory papillomatosis. Immunity may be induced with a vaccine comprising a HPV peptide antigen fused to a viral protein or other antigen. Antibodies and cells may be recovered from an animal (human or otherwise) previously vaccinated with the same vaccine. Of particular interest is the use of HPV L2 peptides designating a neutralizing epitope of HPV.

### DESCRIPTION OF THE PREFERRED EMBODIMENTS

“Antibody” when used in the present application is intended to encompass naturally occurring antibodies (antisera), monoclonal, fragments and derivatives thereof (e.g. Fab, Fab2, etc.), chimeric or reassortant antibodies having plural binding specificities, as well as artificially produced molecules which have binding specificity comparable to natural antibodies (e.g. recombinant antibodies, phage display, single chain antibodies and selected combinatorial library proteins, peptides, nucleic acids and other polymers).

An “anti-HPV” state, response or condition occurs when virus neutralizing antibodies or other immune factors are present that will eliminate, or reduce the number of papillomavirus infections in the upper respiratory tract of the neonate, and reduce the chance that the baby will develop JORRP at some stage later in its life. Likewise, for inducing an “anti-HPV” state, response or condition is older children and even adults.

While more than 60 types of HPV are known, attention has focused on the at least fifteen types which are carcinogenic and are believed to be the cause of cervical cancer. Vaccines against these types have been proposed as a way of preventing cervical cancer. These vaccines generally are based on the L1 neutralizing epitope and are specific for the carcinogenic HPV types. Such vaccines are employed in a traditional manner, immunizing immunocompetent adolescents or adults before exposure.

By contrast, the HPV types causing JRR are typically type 6 or 11, which are not believed to be carcinogenic and are not associated with cervical cancer. In the example of HPV, a fetus or infant is typically exposed in-utero or during delivery. In both situations, advanced vaccination is impractical and due to the immaturity of the immune system, mounting an effective immune response to vaccination would not be expected. Further, because of early exposure, immune tolerance may have already occurred. Therefore, traditional approaches using inactivated virus may not be effectively elicit an effective immune response in such individuals. The present invention involves a vaccine including the L2 neutralizing epitope against these HPV types as a preventative or therapeutic against JRR.

To avoid issues of potential prior tolerance, and because HPV grows poorly in culture, applicants have prepared a vaccine based on the HPV epitope bound to a larger unrelated antigen to which the child is unlikely to have been previously exposed.

A temporary anti-HPV state may be provided to the individual being exposed, or to be exposed to HPV, by administering passive antibodies and/or cellular components from an actively immunized animal. The antibodies are preferably neutralizing. The treatment may be continued periodically for as long as desired, including the entire life of the recipient person.

HPV is also associated with other situations where the immune system is not complete. Head and neck cancers and a number of squamous cell carcinomas in immunocompromised patients undergoing immunosuppressive therapies are also associated with HPV. These individuals may likewise be treated in a similar manner by either active vaccination or passive infusion of HPV neutralizing antibodies and the like.

While an embodiment of the present invention is to induce a protective immune response, it is an other embodiment of the present invention to elicit an immune response to ameliorate the disease by reducing the growth rate or number of tumors thereby reducing the number of surgeries and other therapy needed. This embodiment is performed in the same manner as vaccinating to elicit active immunity.

An alternative method for ameliorating the disease is by transfer of passive antibodies and/or cellular components from another animal to the affected person. The antibodies are preferably neutralizing. If provided early enough or in sufficient amounts, the development of respiratory papillomas may be prevented. Alternatively, by neutralizing HPV, the spread of the disease and formation of new papillomas may be reduced or eliminated thereby reducing the need for as many surgical treatments. Treatment with passive antibodies and/or cellular components may be repeated, even over a lifetime, to maintain an anti-HPV state in the affected person.

While recurrent respiratory papillomatosis (RRP) being discussed is generally of the form of juvenile onset recurrent respiratory papillomatosis (JORRP), the disease may have an adult onset, particularly in immunocompromised individuals. While some treatments involving pregnancy and breast feeding are inappropriate for treatment of adults, the other treatments are applicable for RRP which is not JORRP.

Passive immunity may be provided by periodic injection or infusion of antibodies and/or cellular components every several weeks to few months depending on antibody titer, etc. to maintain a persistent anti-HPV state. The dosages will depend on the age of the recipient and are chosen to maintain a detectable anti-HPV titer in the blood of the recipient.

Passive immunity may also be provided orally or applied to a mucous membrane. Of particular interest is an aerosol containing the passive vaccine to be applied to the respiratory track. In this manner, very high titers of anti-HPV antibodies may be applied to the respiratory track surface to prevent additional infection and papillomas. Aerosol formulations and delivery of protein drugs is well known per se and the antibodies of the present invention may be used in the same manner.

Passive transfer of papillomavirus neutralizing antibodies may be performed by methods other than direct infusion into the recipient. Transplacental transfer of virus neutralizing IgG from the infected mother to her fetus occurs naturally after a neutralizing antibody response is induced in the mother by vaccination starting before or during pregnancy. Transfer of virus-neutralizing IgA from the immunized mother to her neonate via colostrum and breast milk may be performed during lactation to the infant.

Alternatively, the neutralizing antibodies may be administered directly to the mother before, during and after during lactation. In this situation, the antibodies are actually produced by another animal or produced artificially.

It is well established from conventional vaccine knowledge that preexisting neutralizing antibodies can prevent or ameliorate infection. However, in accordance with the present invention, the presence of neutralizing antibodies, either induced or added, can be effective post-infection and may assist in resolution of virus infection, since neutralizing antibodies will prevent reinfection with de novo replicated papillomavirus. Recently, Kawana et al. (2003a) found evidence that natural neutralizing antibodies can be present in neonates of mothers with HPV-6 associated condyloma. These authors found that there were maternally-derived neutralizing antibodies transferred to a newborn of one of two mothers with genital warts. These antibodies appeared to have prevented infection of the neonate with HPV-6.

While not wishing to be bound by any theory, it appears natural boosting maternal neutralizing antibody response by vaccination with antigens that her immune system has been exposed to through infection is occurring. With the maternal immune system may have been effectively primed, further boosting of immunity with L2 peptide vaccines, or L1 VLP vaccines, or L2 protein vaccines of the present invention should increase the titer of neutralizing IgG, primarily IgG1, that is transferred to the fetus transplacentally. Additionally, vaccination according to the present invention should boost maternal IgA neutralizing antibody response by vaccination, preferably via a mucosal route, to boost the level of neutralizing IgA that can be transferred to her breastfeeding newborn via colostrum and breast milk. Note Liu et al, Virology. Dec. 5, 1998;252(1):39-45. Also, direct injection or infusion of anti-HPV neutralizing antibodies into a neonate can prevent or ameliorate virus infection of respiratory mucosal surfaces.

Contrary to the immune response to the L1 protein, peptides derived from the L2 protein of human and rabbit papillomavirus types can generate papillomavirus neutralizing antibodies with a greater spectrum of neutralizing activity than L1 vaccines (Kawana et al., 1999; 2003b; Embers et al., 2003). One embodiment of the present invention uses peptide from the L2 antigen in an attempt to generate neutralizing antibodies against a number of different HPV.

Applicant has expressed cross-neutralizing epitopes from HPV types 6, 11, 16 and 18, in addition to cottontail rabbit papillomavirus and rabbit oral papillomavirus on the surface of tobamovirus particles (K. E. Palmer et al. U.S. patent application Ser. No. 10/654,200, Production of Peptides in Plants as Viral Coat Protein Fusions Filed Sep. 3, 2003). These were used as immunogens in guinea pigs, and found that they are capable of inducing high levels of peptide-specific antibodies.

The vaccines of the present invention are administered parentally, typically by injection e.g. intramuscularly, intradermally or intravenously. The vaccines may also be administered orally or by contact to a mucous membrane. This is particularly preferred when one uses or wishes to induce production of IgA.

Methods for construction of papillomavirus L1, L1/L2 and L2 peptide vaccines are disclosed in the academic and patent literature. Methods for construction of L2 peptide vaccines are disclosed in LSBC U.S. patent filing Ser. No. 10/654,200, Production of Peptides in Plants as Viral Coat Protein Fusions, 3 Sep. 2003. In the present invention, it is preferred to use additional peptides (length 6 to 50 amino acids) that may be useful as peptide vaccines, and as TMV capsid fusion vaccines, and are derived from the sequence of the L2 protein of all HPV mucosal tissue infecting types, preferably HPV-11 and HPV-6. Some specific HPV-11 L2 derived peptides are listed below. Shorter and longer, at least partially overlapping, peptides comprising a part of these peptides may also be used. The peptides should elicit neutralizing antibodies or other effective antiviral response. Homologous peptides from other papillomavirus types may also be used.
HPV-11 L2 N-terminal: ASATQLYQTCKATGTCPPDVIP (SEQ ID NO: 1)
HPV-11 L2 108-120 region: PPLVEPVAPSDPSIVSLIEESAIINAGAPEVVPPTQGF (SEQ ID NO:2)

The underlined sequence has been displayed on the surface of TMV and generates high levels of peptide specific antibodies in vaccinated guinea pigs and mice (K E Palmer et al. U.S. patent application Ser. No. 10/654,200, Production of Peptides in Plants as Viral Coat Protein Fusions, filed 3 Sep. 2003).

### Treatment Protocols/Clinical Trials

Preclinical studies are used with the rabbit oral papillomavirus model and in the cottontail rabbit papillomavirus model (Christensen et al., 2000; Embers et al., 2002). Many of the treatment protocols outlined below may be duplicated in the preclinical model, except that rabbit neonates are challenged with virus soon after delivery. In humans, initial safety trials may occur in non-infected volunteers and in infected, non-pregnant women.

Prior to vaccination, diagnosis of maternal genital HPV infection is made by visual inspection, colcoscopy, PCR based DNA or RNA tests for HPV infection, optional papillomavirus typing or serological analysis for HPV-neutralizing antibodies. HPV-positive mothers are vaccinated with L2:TMV vaccines, or L2 peptide vaccines, or L1 VLP vaccines or L1/L2 VLP vaccines. An adjuvant such as alum may be used. Oral vaccines administered with or without mucosal adjuvant may also be used.

Vaccination of HPV-positive mothers with vaccines delivered mucosally, by intranasal, oral, vaginal or rectal routes to boost IgA production will boost the immune response to neutralize virus by transfer transplacentally, through breast milk, as well as in the maternal genital tract.

The maternal serum and mucosal neutralizing antibody responses may be monitored post vaccination to determine effectiveness or need for additional vaccination. A lack of boosting may necessitate cesarean section delivery.

Neutralizing antibody titers are monitored in cord blood at the time of delivery, and in neonate blood collected after delivery. Additional genetic screening may also be performed. Typically, a PCR based assay for presence of papillomavirus DNA in buccal swabs of newborns is performed, with follow up at 1 week, 1 month and 3 month well baby checks.

### Passive Immune Therapy/Neonate

As before with active immunization, to employ passive immune therapy one typically begins with diagnosis of maternal genital HPV infection by visual inspection, colcoscopy, PCR based DNA or RNA tests for HPV infection with optional papillomavirus typing and serological analysis for HPV-neutralizing antibodies.

The neutralizing antibody titers are monitored in cord blood at the time of delivery and in neonate blood collected after delivery. Genetic screening may be performed simultaneously.

For infants that lack sufficient neutralizing antibodies, an injection or infusion of neutralizing antibody or antibody cocktail is given to newborn.

PCR based assays for presence of papillomavirus DNA or RNA assays from buccal swabs of newborns may be performed with follow up at 1 week, 1 month, 3 month well baby checks. Alternatively one may assay for presence of neutralizing antibody in infant serum.

### Passive Immune Therapy/Pregnant Mother

Passive antibody therapy may be administered to the pregnant or lactating mother before, around and after delivery. Maternal genital HPV infection is diagnosed by visual inspection, colcoscopy, PCR based DNA or RNA tests for HPV infection, optional papillomavirus typing, and serological analysis for HPV-neutralizing antibodies.

When desired or if insufficient neutralizing antibodies are present, an injection or infusion of neutralizing antibodies or antibody cocktail is provided to the mother a few weeks or days prior to delivery. Additional neutralizing antibodies may be provided into the vagina prior to and/or during delivery and/or on the infant immediately after delivery.

Neutralizing antibody titers in cord blood may be monitored at the time of delivery and in the neonate blood collected after delivery. Optional genetic screening may also be performed.

If insufficient neutralizing antibodies are present, an injection or infusion of neutralizing antibody or antibody cocktail in newborn.

PCR based assays for presence of papillomavirus DNA or RNA assays from buccal swabs of newborns, with follow up at 1 week, 1 month, 3 month well baby checks may be performed.

### Maternal Vaccination/Neonate Passive Immune Therapy

A combination of active immunization of the mother according to the above guidelines combined with passive immune therapy of the infant and optionally the mother may be performed. The protocols are essentially a combination of the two above. Either one of the vaccine antigen or the antibody may be conventional with the other one being that of the present invention.

In additional to these protocols, a number of variations may be used. By immunizing or providing antibodies to the mother before delivery one may inherently be treating the fetus with neutralizing antibodies before birth because of transplacental transfer of antibodies (e.g. IgG) neutralizing the papilloma virus. This immunization may be done before or during pregnancy. Alternatively, by treating the mother before, during or after delivery with vaccine that has or induces virus neutralizing antibodies (e.g. secretatory IgA), the antibody is secreted into the colostrum and breast milk for the baby to consume during lactation.

Parental, oral, topical, aerosol, liquid drops or sprays administering virus neutralizing antibodies or derivatives thereof, such as antibody conjugates or fragments (Fab, Fab2) etc. may be provided as the passive immune therapy. The antibodies may be from body fluids, monoclonal antibodies, or antibodies produced by expression of recombinant cells. The antibodies may be protein molecules which resemble antibody molecules only in the binding site such as single chain antibodies. The antibody molecule may be humanized or have an artificial amino acid sequence or glycosylation pattern or be conjugated. These modifications are designed to make the protein more compatible, less antigenic, have a better adsorption, distribution, stability, retention, etc., provided that the basic virus binding or neutralization properties are retained, though the functional activity may be quantitatively changed.

### Construction of a Vaccine

Many different methods for making a suitable antigenic vaccine or antibody-like compound are known per se. Any of these may be used. One preferred embodiment is the method for preparing the vaccines by use of plants as the production host. This embodiment uses an RNA virus vector system producing a transient, cytoplasmic expression that does not rely on stable nuclear integration of the transgene. This technology was previously well established for other uses including high throughput cloning and expressions screening in plants (20) and personalized cancer vaccines (53).

These viral vectors are based on tobacco mosaic virus (TMV) genomes that have been modified to direct expression of foreign genes (60). The basic organization of the TMV genome is 5′-T7 promoter-replicase-subgenomic promoter-movement protein-subgenomic promoter-coat protein-3′. Along with other modifications, an additional subgenomic promoter and an additional gene may be inserted into the genome. Genes of about 2.5 KB of foreign nucleic acids are easily expressible with this system. Typically, these vectors can also express only one cistron, since vectors with a second non-native gene are genetically unstable in plants. TMV invades nearly every cell of the infected plant in a brief 10-14 day time period. By harnessing the highly efficient gene expression capabilities of this virus, heterologous proteins of interest can be synthesized quickly and abundantly. The plant hosts used for such a vector-based production need not used as food crops, nor need they be genetically modified. Instead, only the viral vector has been engineered to deliver the gene of interest to the plant for transient infection. These precautions minimize issues with genetically modified plants, the food supply and potential crossing with other plants and escape of genetic modifications. Alternatively, if one wishes to produce the protein by way of a transgenic plant, animal, yeast or microbial cell, such methods are well known in the art.

When expressing secreted multimeric proteins such as antibodies from such a vector system one may generate a fusion protein (proprotein) joined by the Ustilago maydis virus KP6 killer toxin propeptide sequence as in reference (17). During expression in plants, the proprotein polypeptide is folded and the inter- and intra-chain disulphide bonds are formed. After folding and assembly of the antibody chains, the KP6 propeptide sequence (ProP) is efficiently processed to produce the mature antibody.

The second approach to treatment of HPV infections is the use of HPV-neutralizing antibodies. These antibodies may be produced recombinantly and can be directly manufactured in plants. While a number of HPV-neutralizing monoclonal antibodies exist for research use, these are inappropriate for use for therapeutic or prophylactic purposes. In the present invention, the application of this product is not strictly therapeutic, but prophylactic since it is administered to patients at high risk of giving or receiving an infection. Similarly, HPV-neutralizing antibodies may be administered to babies at risk of developing JORRP, a respiratory infection acquired during birth from maternal HPV-associated genital lesions. Risk of JORRP is 234-times higher in babies born to mothers with clinically evident genital warts (61). Even after respiratory infection, active vaccination or passive immune therapy should ameliorate the disease, its growth rates or its reoccurrences post surgical treatment.

Topical application of neutralizing antibodies to prevent mucosal HPV infection in high-risk infants should also be effective. Application of antibodies to prevent infection of mucosal epithelia with canine oral papillomavirus (COPV) is an model for human mucosa-infecting papillomaviruses used below and provides a disease model establishing credible effectiveness.

For HPV neutralization assays one may use the facile HPV-neutralization assay that relies on generation of pseudoviruses or pseudovirions (PsV) that mimic HPV structure and are derived from L1 and L2 proteins that encapsidate a double-stranded, histone-associated plasmid DNA encoding a reporter gene (secreted alkaline phosphatase, SEAP) (4, 50). This system allows facile measurement of neutralizing antibody titer, and efficiency of neutralization by monoclonal antibodies by assaying for reporter gene activity, and represents a major technical advance in the field.

Previous methods for producing antibodies to a select antigen are known and were used by the applicants to generate single chain antibodies for clinical usage. The process used high throughput cloning, screening and protein manufacturing methods for secreted proteins produced in plants (41, 42, 53, 54). For expression of novel monoclonal antibodies in plants, a proprietary method for expression of two chain antibody genes as single cistrons using the propeptide strategy of a signal peptide-protein domain 1-proprotein-protein domain 2 may be used. The polypeptides are designed so that a disulfide bridge forms between the two protein domains and the proprotein region is later removed by cleavage. This leaves the two protein domains attached by a disulfide bridge, the same configuration as occurs in natural antibodies. Functional monoclonal antibodies may also be produced via conventional transgenic technology, for example (11, 21, 35, 39, 40). Protein accumulation levels in plants of around 1-2 mg/kg are typically achieved. However, when using a single cistron gene sizes of about 1.4 kb, the above mentioned system can easily express >200 mg/kg of recombinant protein.

Lower serum half-lives of Fab or full antibody products may be improved by poly(ethylene glycol) modification technologies. A variety of adjuvants known per se may be used also.

Candidate papillomavirus vaccines based on the L2 protein that have been tested to date in animal models have usually been expressed in bacterial systems, often as glutathione-S-transferase (GST) fusion proteins, and have been purified under denaturing conditions. These L2 antigens induce only low titer antibodies in adults, resulting in poor immune memory and may cause the neutralizing antibody titer to drop below the minimum protective threshold. This problem is particularly acute for JORRP because the fetus/infant has an immature immune system which is likely to provide a weaker immune response.

An embodiment of the present invention is to boost the poor immunogenicity of L2 vaccines by displaying domains of the L2 protein as fusions to self-aggregating carrier proteins. It was previously shown that antigen aggregates tend to function as superior immunogens in comparison with soluble antigens. Domains of proteins displayed in semi-crystalline repetitive arrays, such as on the surface of virus-like particles are known to be particularly immunogenic, since they appear to trigger the innate immune system recognition of pathogen “pattern” (8, 9, 28, 29). In the examples below, immunogenicity is boosted by using short HPV L2 peptides displayed on the surface of TMV particles. The size of peptides that may be displayed in this system was previously limited to short peptides of approximately 20-25 amino acids, which may not be sufficient to recreate conformational epitopes characterized by longer stretches of amino acids that are necessary for proper protein folding. In the present invention, one may test larger domains of L2 as fusions to self-aggregating carrier proteins, as the quality of the immune response is likely to be improved in comparison with shorter peptides.

Many self-aggregating viral capsid proteins (cp) have been expressed, but in a preferred embodiment, two proteins are of particular interest as antigen display carriers since they have proven capacity to display peptides as large as whole proteins on their surfaces: hepatitis B core antigen HBcAg and the coat protein (cp) of potato virus X (PVX). Both of these proteins can display the green fluorescent protein (GFP) on their surface without abolishing capsid formation (15, 36, 52). When expressed in plants, the HBcAg particles accumulate to high level and can easily be purified. The PVX particle is a long, filamentous structure. Like TMV, it is constructed from subunits composed of two-layer disk structures that can tolerate fusions with quite large proteins including antibody fragments. These structures have shown superior immunogenicity over unfused vaccines (59). A third particulate carrier protein that is capable of self-assembly when expressed recombinantly is the E2 acetyltransferase scaffold component of the thermophilic bacterium Bacillus stearothermophilus. A 28 kDa C-terminal domain of E2 is capable of self-assembly into an icosahedral cage structure composed of 60 m copies of E2. Under natural conditions the E2 icosahedron is linked tightly, but not covalently, with two other enzymes—a specific 2-oxo acid decarboxylase (E1) and a dihydrolipoyl dehydrogenase (E3). Domingo et al. (16) showed that the E2 core domain may be linked to various peptides and proteins as large as GFP, to form stable, particulate structures that are highly immunogenic. The E2 scaffold is highly thermostable, a property that could prove very useful for purification of E2 fusion structures.

The present invention may use any of these particulate carrier proteins to display a large domain of the L2 protein. Although the literature predicts that fusion of proteins or peptides to virus-like particle carriers should enhance the immunogenicity of the fused domain, this is untested for L2. There appears to be a domain of L2 that induces cross-neutralizing antibodies in vaccinated animals. However, it is not certain that this domain will be accurately displayed on any of the carrier proteins.

### Pseudovirion Production and Qualification of the PsV Neutralization Assay

The methods for production of papillomavirus PsV are well described in two recent publications (4, 50). In the present invention testing of PsV of HPV-16 and HPV-18 that encapsidate a reporter plasmid encoding secreted alkaline phosphatase (SEAP) may be used. In order to assay for breadth of neutralization activity of plant-produced antibodies and serum of animals vaccination with the present invention's vaccine antigens, one may test PsV of COPV and from different HPV subgroups. In addition to HPV-16 (group A9) and HPV-18 (A7), HPV-11 (A10) was chosen, due to its involvement in the JORRP disease, and HPV-51 (A5), a high-risk type. Also PsV of high-risk HPV types 31 and 45, as these are in the same groups as HPV-16 and HPV-18, respectively, and offer the potential for assaying for breadth of neutralization activity both within and between groups.

### Construction of New Papillomavirus PsV.

The pseudoviral particles are purified by ultracentrifugation through OptiPrep (Sigma) gradients, as described (4, 50). Use of genes encoding L1 and L2 with optimal codon usage for expression in mammalian cells is used (4), and may necessitate construction of synthetic genes encoding structural genes of HPV types 11, 31, 45, 51 and COPV. Synthetic genes may be purchased from DNA 2.0 (Palo Alto, Calif.). These genes are inserted into expression plasmids p16Lh and p16L2h (4) in place of the HPV16L1 and L2 genes, respectively. PsV are generated by co-transfection of 293TT cells with the appropriate L1 and L2 expression constructs, and reporter plasmid pYSEAP. The quality of PsV is analyzed by SDS-PAGE and electron microscopy. The titer of functional PsV is determined by infection of 293TT cells and measurement of SEAP activity in supernatants of transfected cells, as per (50).

### Qualification of PsV Neutralization Assay:

Metrics to validate all PsV will be: (1) visualization of virus-like particles with typical papillomavirus morphology by electron microscopy; (2) detection of reporter gene activity in supernatants of 293TT cells infected with PsV and (3) positive control sera must neutralize the homologous PsV at a titer of no less than 1000. Neutralization titers are defined as the reciprocal of the dilution of antibody necessary to achieve 50% inhibition of the amount of SEAP activity in cells infected with PsV. The neutralization criterion validates both PsV and the quality of test sera. For COPV and HPV-11 one may validate neutralization activity of pre-existing antibody compositions such as monoclonal antibodies known to bind conformational epitopes (COPV and HPV-11) and to neutralize authentic HPV-11 (mAb H11.B2 (14), Chemicon Inc).

Methods for construction and validation of papillomavirus PsV are established in the literature (4, 50). Nonetheless, Buck et al. (4) experienced some difficulty in generating PsV for bovine papillomavirus, and showed that low yield could be mitigated by modification of both codon usage and RNA sequences that cause mRNA instability (58, 63). Construction of synthetic DNA sequences that conform to common codon usage rules for highly expressed human genes should avoid this problem.

### Demonstration That Produced Antibodies Effective Against Papillomavirus Infection.

Papillomaviruses are very effectively neutralized in vitro by monoclonal antibodies targeted to epitopes in both the L1 and L2 structural proteins (12-14, 23, 56, 57, 70). While monoclonal antibodies have not been used in passive immunization it was shown that passive serum transfer from vaccinated and previously infected dogs can protect naïve animals from virus challenge (24, 67). A number of mouse monoclonal antibodies are capable of potent neutralization of HPV in vitro, for example H11.B2 neutralizes HPV-11 and H16.V5 neutralizes HPV-16 (14, 26, 55, 70). A set of monoclonal antibodies were used that were directed against COPV VLPs; most of these recognize only VLPs, not disrupted particles and hence are directed against conformational epitopes. In general, it appears that most monoclonal antibodies with potent HPV-neutralizing activity directed at the L1 capsid protein recognize conformational epitopes. Due to difficulties in obtaining sufficient quantities of authentic virus, these monoclonal antibodies were generated against VLPs, not authentic virions, and that L2 was not present in the recombinant VLP immunogens. Another important point is that not all VLP-reactive monoclonal antibodies recognize authentic virus (25), which implies that virions or pseudovirions have subtly different structures to VLPs.

To address these problems, one may first validate the concept that plant viral vector-produced monoclonal antibodies, Fabs or scFv can have biological activity in vitro, as measured in the PsV neutralization assay, and that these antibodies might also have protective effects in vivo, as measured in the dog-COPV challenge model. The assay used to determine broad spectrum neutralization involves screening various antibody molecules produced against multiple papillomavirus targets to discover new antibodies that neutralize two target papillomaviruses: (1) the model papillomavirus COPV and (2) HPV-11, which plays an important role in the etiology of JORRP.

Expression of COPV mAbs in Plants and Validation of In Vitro and In Vivo Activity of Plant-Produced mAbs:

A panel of COPV monoclonal antibodies expressed in plants that have been pre-screened for reactivity with VLPs is used. Previously, we developed robust methods for amplification and identification of novel immunoglobulin sequences from human lymphatic tissue. Other known methods of random or site specific mutagenesis may be used to generate an even more diverse population of antibody sequences. Nucleic acid shuffling techniques may also be used such as Genetic Reassortment by Mismatch Resolution (GRAMMR), covered by LSBC patent applications (44-49).

Here, the heavy chain, light chain and KP6 propeptide genes are PCR amplified with oligonucleotides that overlap by 20 nucleotides at the junction sites and assembled in a one-step PCR reaction to create the light chain-KP6 propeptide-heavy chain gene fusion. The PCR amplified fragments are inserted into the TMV vectors described above. Both monoclonal antibody and Fab-cistron clones for each COPV-reactive monoclonal antibody are prepared.

Recombinant viral constructs containing the antibody assemblies are transcribed in vitro to generate infectious RNA. Nicotiana benthamiana plants are inoculated with infectious transcripts and infection of plants scored visually. At 9 to 12 days post-inoculation, first-round screening of infected plants involves evaluation of secreted proteins contained in the apoplast by extraction of the interstitial fluid (IF) according to methods we have published previously (41, 42). The proteins are separated by reducing and non-reducing SDS-polyacrylamide gel electrophoresis (PAGE) and stained with Coomassie brilliant blue for the detection and sizing of novel proteins. ELISAs with COPV VLPs used as capture antigen are performed on extracts to verify the presence of VLP-reactive material. Constructs that produce VLP-reactive material in the IF are identified for further investigation, including DNA sequencing and analysis of neutralization activities as described above. At least two mAb-expressing constructs and two Fab-expressing constructs are screened further, with partial purification of immunoglobulins so that concentrations of antibodies can be semi-standardized to allow fair comparisons between molecules.

To obtain purified immunoglobulins, larger numbers of plants are inoculated, and antibodies purified by methods described previously. Affinity chromatography with Protein A or Protein G provides substantial purification for most antibodies we have expressed in plants. Since Fabs lack the Fc regions necessary for recognition by Protein G or Protein A, they are purified based on their biochemical properties. All chromatography steps are performed with an AKTA Purifier system (Amersham). Additional affinity purification with immobilized HPV L2 antigens or an epitope may optionally be used.

As controls, mammalian cell produced monoclonal antibodies and Fab antibodies are made from the parental hybridoma lines from which the recombinant or plant-produced monoclonal antibodies and Fabs were derived. These antibodies are purified either from culture supernatants, or from ascites fluid, produced using standard methods, such as those under contract to Antibodies Inc. (Davis Calif.). The monoclonal antibodies are further purified by affinity chromatography as described above. To produce Fabs, purified monoclonal antibody molecules are digested with immobilized papain, and purified by subtraction of the cleaved Fc region by Protein G chromatography. If necessary, further chromatographic steps to achieve purity of greater than 50%, as determined by Coomassie brilliant blue stained SDS-PAGE and densitometry may be used. The protein concentration in each sample is determined using a BCA protein assay, and the size of purified molecules verified by MALDI-TOF mass analysis.

The ability of purified antibodies to recognize COPV PsV is verified by antigen capture ELISA according to methods described previously (64, 65). The affinities of the different immunoglobulins for the COPV PsV are measured by surface plasmon resonance (SPR) biosensor technology on a Biacore X instrument. Briefly, PsV are bound to activated biosensor chips (BIAcore). Purified antibodies or Fabs are injected onto the PsV-coupled biosensor chips. Dissociation constants (K_{D}) derived and used to compare binding affinities of different antibodies. The K_{D} is a useful metric to use to compare different antibodies, with lower K_{D} indicative of stronger binding of the antibody to the target. This allows one to compare binding affinities of plant and mammalian cell produced molecules head-to-head. The PsV neutralization assay to characterize the biological activity of plant-produced molecules in comparison with the cognate hybridoma-derived version is used as the determination of actual effectiveness in neutralization once the population of antibodies has been reduced to a small number.

To generate data showing that plant-produced monoclonal antibodies and Fabs or scFvs can have biological activity in vivo, one may use the dog-COPV challenge model characterized by Drs. Jenson and Ghim (JGBCC) (3, 24, 67). Briefly, weanling beagles (six weeks old) are infused intravenously with 100 micrograms per kilogram of body mass of plant produced and control antibodies. The dogs are challenged with COPV the next day by abrasion of the dorsal and maxillary mucosa with a wire brush, followed by application of an infectious COPV stock derived from a wart homogenate previously qualified for infectivity (24, 67). The control group receives HPV-11 neutralizing mAb, which is not expected to neutralize COPV. However, sera from these dogs optionally may be used to evaluate serum stability of the antibody over time, thus generating useful data from the control group. If Fab molecules show poor neutralizing activity in vitro that is greater than one order of magnitude lower than the parental mAb, groups that test Fabs will be deleted from further study because poor in vitro neutralization activity will probably translate into reduced clinical efficacy. The measure success of the dog passive immunization experiment is protection of dogs that receive plant-produced antibodies from infection with COPV. Partial protection from COPV challenge, evidenced by reduced lesion size or number in experimental groups relative to controls is also considered success.

### Discovery and Plant Expression of New Papillomavirus-Neutralizing Antibodies:

Rabbit monoclonal antibodies (RabMAbs) have a number of important advantages over mouse monoclonal antibodies (mAbs). Rabbit antisera are generally of higher affinity than the equivalent mouse antisera, and RabMAbs often exceed the binding affinity of mouse mAbs; there is also higher homology between rabbit and human immunoglobulins in the scaffold regions, making RabMAbs easier to humanize, and hence develop as therapeutic products. For example, Epitomics Inc. (Burlingame Calif.) offers a RabMAb production. Data from (25) show that L1 VLPs differ subtly in their antigenicity relative to native virions and, given that PsV are capable of infecting cells mediated by L2 (4), it appeared likely that one can generate RabMAbs with improved binding affinity and perhaps neutralization activity against papillomaviruses by this strategy.

100 micrograms of pseudovirions of COPV and HPV-11 manufactured according to methods described by Buck et al. (4) is injected into each rabbit. Approximately 4,000 hybridomas are screened for PsV binding activity. L2 proteins as histidine-tagged antigens in mammalian cells, are previously prepared and purified by immobilized metal affinity chromatography (IMAC, Qiagen). The L2 protein is used for screening the same approximately 4,000 hybridomas. Thus, by screening with PsV hybridomas that react with conformational epitopes on PsV are obtained. These antibodies are usually against the major capsid gene, thus necessitating a second screening against HPV L2 alone to select for cells secreting antibodies reactive with L2 epitopes that are surface exposed. Blocking L2 interaction with a presumptive secondary receptor on the surface of cells (31, 68, 71) is the presumptive mechanism for neutralizing papillomavirus infection.

RabMAbs are screened by ELISA and by PsV neutralization assay as described above. The 20 most promising supernatants of hybridomas that secrete mAbs reactive against each of the target antigens (COPV and HPV-11 PsV and L2 proteins) are recovered. The immunoglobulins genes from each hybridoma are cloned and expressed inn plants according to the methods described above. Once again, VLP-binding and PsV neutralization assays are used in the screen for plant-produced immunoglobulins with papillomavirus-neutralizing activity. Constructs encoding the two or three molecules that appear to have the strongest neutralization activity are scaled up, and immunoglobulins purified from these plants, according to the methods described above.

It is probable that anti-COPV and anti-HPV-11 RabMAbs that bind L1 have binding sites that overlap those of extant mouse-derived neutralizing antibodies. Competitive ELISA assays, where one antibody is labeled with biotin and detected with horseradish peroxidase-labeled streptavidin and the other unlabeled are used to determine whether antibody binding sites on PsV overlap, and whether binding of L2-specific antibodies is inhibited or enhanced by prior L1 binding, and vice versa. Similarly, SPR/Biacore analyses may be used in pairwise competitive binding analyses as described (70). In order to characterize the RabMAbs further the binding affinities are measured by SPR in comparison with mouse mAbs, according to the methods described above.

The plant produced recombinant antibodies are used to demonstrate protection against COPV challenge in dogs infused with an L1 VLP-reactive monoclonal antibody or antibody fragment, for ethical reasons we restrict a second dog trial to L2-reactive RabMAbs only. The results are still valid to prove that L2-reactive mAbs also protect against papillomavirus infection. Alternatively, L1-reactive RabMAbs are tested in vitro HPV neutralization analyses to prove that plant-produced RabMAbs have enhanced activity over pre-existing mouse mAbs.

### Metrics Used for Evaluation of Success

| Subtask | Metrics used for evaluation of success |
|---|---|
| Production of COPV-and | (1) Reaction of IF proteins to VLPs in ELISA. |
| HPV-reactive mAbs | |
| Characterization of mAbs | Physical characterization: DNA sequence and molecular mass |
| and RabMAbs | Neutralization of appropriate PsV |
| | Description of binding site, i.e. overlaps or does not overlap with |
| | another mAb |
| | Determination of K_{D} by SPR/Biacore |
| Purification of | Greater than 50% purity by SDS-PAGE and densitometry |
| immunoglobulins | |
| Discovery of RabMAb | Neutralization efficiency of COPV RabMAb produced in plants |
| with improved biological | exceeds the neutralization efficiency of the best mAb produced in |
| activity | plants, as measured by end point neutralization titer in PsV |
| | neutralization assay |
| | Neutralization efficiency of HPV-11 RabMAb exceeds that of |
| | control mAb H11.B2 (10) |
| | RabMAb reactive with HPV-11 L2 neutralizes HPV-11 and one |
| | other HPV type. |
| In vivo neutralization by | At least partial protection of dogs challenged with COPV that is |
| plant produced COPV | mediated by infusion of COPV-reactive monoclonal antibody. |
| antibody | Partial protection is defined as reduced lesion size or number in |
| | experimental animal group, relative to control animals. |
| | Complete protection against COPV challenge in animals that |
| | receive infusion of COPV-reactive monoclonal antibody/ies. |

### Development of Immunogens That Induce Broadly Neutralizing Antibodies and Protect Against Papillomavirus Infection

In this example, three different particulate carrier proteins are used to display domains of the L2 protein of two different papillomaviruses, COPV and HPV-11.

Analysis of Expression of B. stearothermophilus E2 Particles in Plants:

B. stearothermophilus E2 core protein and a synthetic gene encoding the 28 kDa E2 sequence (Pro174-Ala428; SwissProt accession number P11961) that conforms to tobacco-preferred codon usage are constructed. This gene is inserted into TMV GENEWARE® vectors and DNA sequence verified. N. benthamiana plants are inoculated with infectious transcripts, and symptoms monitored. At various time points, 7 to 15 days post infection, leaf disks are punched from infected plant tissue and analyzed by SDS-PAGE, where accumulation of a novel protein of approximately 28 kDa indicates that the E2 protein has been expressed successfully. The particulate proteins are precipitated from the plant extracts by addition of polyethylene glycol (6000) to 10% in the presence of salt, followed by centrifugation, following the methods previously described in U.S. Pat. No. 6,730,306. The molecular mass of the precipitated proteins will be confirmed by MALDI-TOF and their particulate nature verified by electron microscopy.

### Construction and Purification of Recombinant L2 Fusion Proteins:

The locations of the binding epitopes on L2 has been proposed (33, 62). The synthetic genes containing the N-terminal 180 amino acids of this approximately 500 amino acid protein, with linear epitopes around amino acids 69-81 and 108-120 are constructed using the methods described above to derive sequences for fusion to three different particulate carrier proteins: 1) HBcAg; 2) PVX cp and 3) B. stearothermophilus E2. The first two carrier proteins express at high level in plants via GENEWARE® vectors in the inventors lab. Protein fusions placed at the N-terminus are typically used. The L2 protein contains nuclear localization sequences (NLS) at both termini (66). Constructs are prepared for all fusions without the putative NLS, predicted to be within the first 10 amino acids of L2. Sequences encoding from 50 to 200 of amino acids of COPV L2 between amino acids 9 to 229, and a similar domain of the L2 protein of HPV type 11 are amplified by PCR and fused to particulate carrier proteins.

The HBcAg protein tolerates insertion of foreign sequences at three different points: the N-terminus, within the surface spike—the major immunodominant region (MIR)—and at the C-terminus. Chimeric core antigen capsid displaying foreign amino acids increased the immunogenicity of the grafted proteins substantially (36, 52). A library of L2 amino acid sequences encoding the entire 200 amino acid sequence is inserted, and smaller overlapping domains of 100 amino acids (3 constructs for each of COPV and HPV-11), or 50 amino acids (7 constructs each) in the MIR between amino acids 28 and 32. Recombinant proteins that accumulate to Coomassie blue-stainable levels in total protein extracts from infected plants are scaled up.

The PVX coat protein also tolerates fusion of large peptide domains—at the N-terminus (15). The native PVX cp accumulates to reasonably high level (about 200 mg/kg infected plant material) when expressed in N. benthamiana via GENEWARE®. A library of L2 fusions to the N-terminus of PVX is generated, as described above for HBcAg. Similarly, L2 fusions to the N-terminus of E2 are constructed. In all cases, expression of proteins is evaluated by SDS-PAGE, western blot with appropriate antisera and/or electron microscopy.

HBcAg VLPs are purified in plants using standard methods applicable to other icosahedral particles expressed in planta. Briefly, the harvested plant material is extracted in 2 volumes of a 50 mM acetate buffer containing antioxidant, and adjusted to pH 5. At this pH the fraction 1 (F1) proteins and associated pigment coagulate, and removed by centrifugation. The clarified supernatant is then adjusted to an alkaline pH between 8.5 and 9.5, and the resulting non-proteinaceous precipitate removed by centrifugation. The supernatant is contacted with 3-5% weight per volume activated carbon. Under alkaline conditions, the overall VLP surface charge is negative. This, combined with the macromolecular structure of particles such as HBcAg, results in exclusion of the majority of the particles from the negatively charged pores of the activated carbon. In contrast, plant-derived globular proteins can diffuse freely into the pores and are retained by short-range attractive Van der Waals forces. Using activated carbon removal of contaminating proteins, substantial purification of the HBcAg is obtained. The principal impurity, TMV particles, is also excluded. The level of host protein removal from the solution is adjusted by changing the buffer conductivity: addition of salt improves host protein adsorption to the activated Carbon by neutralizing ionic repulsions. However, this increased purity must be balanced against higher losses of the icosahedral structures. Following the activated carbon treatment, differential precipitation with polyethylene glycol is used to remove the majority of the TMV and chromatography on hydroxyapatite resin is performed as a polishing step. This procedure yields HBcAg particles at greater than 95% purity with a process recovery of 30%, which compares favorably to bacterial expression where recoveries of 3-10% are reported. This method is used (with minor adaptations) to HBcAg, E2 and PVX cp fusions.

In the constructs of the present invention, it is preferred to optimize codon usage for expression in plants. Codon usage has not posed significant problems for protein expression for many other genes in the past and is used here to eliminate this variable when designing synthetic genes. Targeting the recombinant protein to a specific subcellular compartment may be employed to solve protein degradation in planta. Plant proteases that are implicated in protein degradation post-extraction can be inactivated by addition of protease inhibitors, by the use of heat during the extraction process, by altering the pH of the extract out of the protease's active range, or by a combination of these approaches. Recombinant vaccine candidates are qualified by SDS-PAGE (purity and concentration), MALDI-TOF and if necessary MALDI-TOF of tryptic fragments (identity), protein concentration, and by endotoxin testing prior to use as vaccines.

Immunization of mice and rabbits with candidate L2 vaccines: BALB/c mice and New Zealand White rabbits are used to evaluate immunogenicity of particulate L2 fusion proteins purified from plants. For cost and ease-of-use considerations, immunization of mice first is used as the first screen for immunogenicity of candidate vaccines. The His-tagged COPV and HPV-11 L2 proteins expressed are produced as above and are used for coating of ELISA plates, as well as for a control non-particulate antigen in rabbit immunization experiments. Compositions that show enhanced immunogenicity in mice in comparison with the L2 protein controls are scaled up and used in rabbit trials. One group of rabbits receives the gold standard vaccine (COPV and HPV-11 VLPs) purified from insect cells to serve as a control. This vaccine produces antibodies that neutralize only COPV and HPV-11 in the PsV neutralization assay. Neutralizing antibodies produced by the method of the present invention are assayed for improved breadth of neutralization activity in comparison with L1 VLP vaccine controls, as described above.

### COPV Vaccination and Challenge Trial:

Six week old weanling beagle dogs are immunized with the COPV-L2 vaccine antigen that produced the highest titer of COPV-neutralizing antibodies above. Control cohorts receive COPV L1 VLPs expressed in and purified from insect cells (positive control) and HPV-11 L2 particulate fusion vaccines purified from plants (negative control). On completion of the vaccine series, animals are challenged with infectious COPV and monitored for the appearance of lesions. Partial protection against COPV challenge in the L2 fusion vaccine group qualifies as success.

Demonstration that a plant-produced L2 fusion protein vaccine can provide protection against papillomavirus challenge.

### Metrics Used for Evaluation of Success

| Subtask | Metrics used for evaluation of success |
|---|---|
| Expression of E2 particles | Accumulation of appropriate sized protein (28 kDa) at levels |
| in plants | greater than 10 mg/kg fresh weight of plant material. |
| | Assembly of recombinant E2 protein into particulate structures |
| | visualized by electron microscopy |
| Construction and | Production and purification of at least one particulate COPV L2- |
| purification of recombinant | fusion protein and at least one HPV-11 L2 fusion protein. |
| L2 fusion proteins | Recombinant vaccine products pass quality criteria for purity |
| | (greater than 50%) and identity (correct molecular mass by |
| | MALDI-TOF) |
| Immunogenicity of | Animals immunized with recombinant vaccine candidates produce |
| recombinant L2 vaccines | L2-reactive antibodies, assayed by ELISA. |
| | Sera from animals neutralize homologous virus at titer of >100 |
| | Sera from animals immunized with HPV-11 L2 vaccines neutralize |
| | at least one heterologous HPV type at improved titer in comparison |
| | with L1 VLP vaccines. |
| COPV vaccination and | Particulate COPV L2 vaccine is immunogenic in dogs, measured |
| challenge trial | by L2-reactive antibodies in ELISA |
| | Dogs immunized with particulate L2 vaccines are at least partially |
| | protected from challenge with COPV. |

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

### REFERENCES

1. Altman, L. K. 2004. Action on Diseases in Women Is Urged, p. 13A, New York Times, Saturday, February 28^{th}, Late Final ed, New York.

2. Anon. 2004. Human Papillomavirus: Vaccine, then antiviral? Datamonitor Healthcare Report BFHC0643, New York.

3. Bell, J. A., J. P. Sundberg, S. J. Ghim, J. Newsome, A. B. Jenson, and R. Schlegel. 1994. A formalin-inactivated vaccine protects against mucosal papillomavirus infection: a canine model. Pathobiology 62:194-8.

4. Buck, C. B., D. V. Pastrana, D. R. Lowy, and J. T. Schiller. 2004. Efficient intracellular assembly of papillomaviral vectors. J Virol 78:751-7.

5. Campo M S (2002) Animal models of papillomavirus pathogenesis. Virus Research 89:249-61.

6. Campo, M. S. 1997. Vaccination against papillomavirus in cattle. Clin Dermatol 15:275-83.

7. Campo, M. S., B. W. O'Neil, G. J. Grindlay, F. Curtis, G. Knowles, and L. Chandrachud. 1997. A peptide encoding a B-cell epitope from the N-terminus of the capsid protein L2 of bovine papillomavirus-4 prevents disease. Virology 234:261-6.

8. Campo, M. S., G. J. Grindlay, B. W. O'Neil, L. M. Chandrachud, G. M. McGarvie, and W. F. Jarrett. 1993. Prophylactic and therapeutic vaccination against a mucosal papillomavirus. J Gen Virol 74 (Pt 6):945-53.

9. Chackerian, B., L. Briglio, P. S. Albert, D. R. Lowy, and J. T. Schiller. 2004. Induction of Autoantibodies to CCR5 in Macaques and Subsequent Effects upon Challenge with an R5-Tropic Simian/Human Immunodeficiency Virus. J Virol 78:4037-47.

10. Chackerian, B., P. Lenz, D. R. Lowy, and J. T. Schiller. 2002. Determinants of autoantibody induction by conjugated papillomavirus virus-like particles. J Immunol 169:6120-6.

11. Chandrachud, L. M., G. J. Grindlay, G. M. McGarvie, B. W. O'Neil, E. R. Wagner, W. F. Jarrett, and M. S. Campo. 1995. Vaccination of cattle with the N-terminus of L2 is necessary and sufficient for preventing infection by bovine papillomavirus-4. Virology 211:204-8.

12. Chargelegue, D., N. D. Vine, C. J. van Dolleweerd, P. M. Drake, and J. K. Ma. 2000. A murine monoclonal antibody produced in transgenic plants with plant-specific glycans is not immunogenic in mice. Transgenic Res 9:187-94.

13. Christensen N D, Cladel N M, Reed C A, Han R (2000) Rabbit oral papillomavirus complete genome sequence and immunity following genital infection. Virology 269: 451-61.

14. Christensen, N. D., and J. W. Kreider. 1991. Neutralization of CRPV infectivity by monoclonal antibodies that identify conformational epitopes on intact virions. Virus Res 21:169-79.

15. Christensen, N. D., and J. W. Kreider. 1993. Monoclonal antibody neutralization of BPV-1. Virus Res 28:195-202.

16. Christensen, N. D., J. W. Kreider, N. M. Cladel, S. D. Patrick, and P. A. Welsh. 1990. Monoclonal antibody-mediated neutralization of infectious human papillomavirus type 11. J Virol 64:5678-81.

17. Cruz, S. S., S. Chapman, A. G. Roberts, I. M. Roberts, D. A. Prior, and K. J. Oparka. 1996. Assembly and movement of a plant virus carrying a green fluorescent protein overcoat. Proc Natl Acad Sci USA 93:6286-90.

18. Domingo, G. J., S. Orru, and R. N. Perham. 2001. Multiple display of peptides and proteins on a macromolecular scaffold derived from a multienzyme complex. J Mol Biol 305:259-67.

19. Edwards, P., M. Fronefield, L. Hamm, and S. J. Reinl. 2003. Presented at the American Society for Virology Annual Meeting, Davis, Calif., July 2003.

20. Embers M E Budgeon L R, Culp T D, Reed C A, Pickel M D, Christensen N D (2003) Differential antibody responses to a distinct region of human papillomavirus minor capsid proteins Vaccine (in press).

21. Embers M E, Budgeon L R, Pickel M, Christensen N D (2002) Protective immunity to rabbit oral and cutaneous papillomaviruses by immunization with short peptides of L2, the minor capsid protein. J Virol. 76:9798-805.

22. Embers, M. E., L. R. Budgeon, M. Pickel, and N. D. Christensen. 2002. Protective immunity to rabbit oral and cutaneous papillomaviruses by immunization with short peptides of L2, the minor capsid protein. J Virol 76:9798-805.

23. Embers, M. E., L. R. Budgeon, T. D. Culp, C. A. Reed, M. D. Pickel, and N. D. Christensen. 2004. Differential antibody responses to a distinct region of human papillomavirus minor capsid proteins. Vaccine 22:670-80.

24. Emeny R T, Wheeler C M, Jansen K U, Hunt W C, Fu T-M, Smith J F, MacMullen S, Esser M T, Paliard X (2002) Priming of human papillomavirus type 11-specific humoral and cellular immune responses in college-aged women with a virus-like particle vaccine. J. Virol. 76:7832-7842.

25. Fitzmaurice, W. P., S. Holzberg, J. A. Lindbo, H. S. Padgett, K. E. Palmer, G. M. Wolfe, and G. P. Pogue. 2002. Epigenetic modification of plants with systemic RNA viruses. Omics 6:137-51.

26. Frigerio, L., N. D. Vine, E. Pedrazzini, M. B. Hein, F. Wang, J. K. Ma, and A. Vitale. 2000. Assembly, secretion, and vacuolar delivery of a hybrid immunoglobulin in plants. Plant Physiol 123:1483-94.

27. Gaukroger, J. M., L. M. Chandrachud, B. W. O'Neil, G. J. Grindlay, G. Knowles, and M. S. Campo. 1996. Vaccination of cattle with bovine papillomavirus type 4 L2 elicits the production of virus-neutralizing antibodies. J Gen Virol 77 (Pt 7):1577-83.

28. Gelder C M, Williams O M, Hart K W, Wall S, Williams G, Ingrams D, Bull P, Bunce M, Welsh K, Marshall S E F, Borysiewicz L (2003) HLA Class II polymorphisms and susceptibility to recurrent respiratory papillomatosis. J. Virol. 77: 1927-1939.

29. Ghim, S. J., R. Young, and A. B. Jenson. 1996. Antigenicity of bovine papillomavirus type 1 (BPV-1) L1 virus-like particles compared with that of intact BPV-1 virions. J Gen Virol 77 (Pt 2):183-8.

30. Ghim, S., J. Newsome, J. Bell, J. P. Sundberg, R. Schlegel, and A. B. Jenson. 2000. Spontaneously regressing oral papillomas induce systemic antibodies that neutralize canine oral papillomavirus. Exp Mol Pathol 68:147-51.

31. Ghim, S., N. D. Christensen, J. W. Kreider, and A. B. Jenson. 1991. Comparison of neutralization of BPV-1 infection of C127 cells and bovine fetal skin xenografts. Int J Cancer 49:285-9.

32. Hines, J. F., S. J. Ghim, N. D. Christensen, J. W. Kreider, W. A. Barnes, R. Schlegel, and A. B. Jenson. 1994. Role of conformational epitopes expressed by human papillomavirus major capsid proteins in the serologic detection of infection and prophylactic vaccination. Gynecol Oncol 55:13-20.

33. Jahan-Parwar, B., D. K. Chhetri, S. Hart, S. Bhuta, and G. S. Berke. 2003. Development of a canine model for recurrent respiratory papillomatosis. Ann Otol Rhinol Laryngol 112:1011-3.

34. Jegerlehner, A., A. Tissot, F. Lechner, P. Sebbel, I. Erdmann, T. Kundig, T. Bachi, T. Storni, G. Jennings, P. Pumpens, W. A. Renner, and M. F. Bachmann. 2002. A molecular assembly system that renders antigens of choice highly repetitive for induction of protective B cell responses. Vaccine 20:3104-12.

35. Jegerlehner, A., T. Storni, G. Lipowsky, M. Schmid, P. Pumpens, and M. F. Bachmann. 2002. Regulation of IgG antibody responses by epitope density and CD21-mediated costimulation. Eur J Immunol 32:3305-14.

36. Kawana K, Kawana Y, Yoshikawa H, Taketani Y, Yoshiike K, Kanda T. (2001) Nasal immunization of mice with peptide having a cross-neutralization epitope on minor capsid protein L2 of human papillomavirus type 16 elicit systemic and mucosal antibodies. Vaccine. 19:1496-502.

37. Kawana K, Yasugi T, Kanda T, Kawana Y, Hirai Y, Yoshikawa H, Taketani Y (2002) Neutralizing antibodies against oncogenic human papillomavirus as a possible determinant of the fate of low-grade cervical intraepithelial neoplasia. Biochem Biophys Res Commun. 296:102-5.

38. Kawana K, Yasugi T, Kanda T, Kino N, Oda K, Okada S, Kawana Y, Nei T, Takada T, Toyoshima S, Tsuchiya A, Kondo K, Yoshikawa H, Tsutsumi O, Taketani Y (2003b) Safety and immunogenicity of a peptide containing the cross-neutralization epitope of HPV16 L2 administered nasally in healthy volunteers. Vaccine 21:4256-60.

39. Kawana K, Yasugi T, Yoshikawa H, Kawana Y, Matsumoto K, Nakagawa S, Onda T, Kikuchi A, Fujii T, Kanda T, Taketani Y. (2003a) Evidence for the presence of neutralizing antibodies against human papillomavirus type 6 in infants born to mothers with condyloma acuminata. Am J Perinatol 20:11-6.

40. Kawana K, Yoshikawa H, Taketani Y, Yoshiike K, Kanda T (1999) Common neutralization epitope in minor capsid protein L2 of human papillomavirus types 16 and 6. J Virol. 73:6188-90.

41. Kawana, K., H. Yoshikawa, Y. Taketani, K. Yoshiike, and T. Kanda. 1999. Common neutralization epitope in minor capsid protein L2 of human papillomavirus types 16 and 6. J Virol 73:6188-90.

42. Kawana, K., K. Matsumoto, H. Yoshikawa, Y. Taketani, T. Kawana, K. Yoshiike, and T. Kanda. 1998. A surface immunodeterminant of human papillomavirus type 16 minor capsid protein L2. Virology 245:353-9.

43. Kawana, K., T. Yasugi, T. Kanda, N. Kino, K. Oda, S. Okada, Y. Kawana, T. Nei, T. Takada, S. Toyoshima, A. Tsuchiya, K. Kondo, H. Yoshikawa, O. Tsutsumi, and Y. Taketani. 2003. Safety and immunogenicity of a peptide containing the cross-neutralization epitope of HPV16 L2 administered nasally in healthy volunteers. Vaccine 21:4256-60.

44. Kawana, K., Y. Kawana, H. Yoshikawa, Y. Taketani, K. Yoshiike, and T. Kanda. 2001. Nasal immunization of mice with peptide having a cross-neutralization epitope on minor capsid protein L2 of human papillomavirus type 16 elicit systemic and mucosal antibodies. Vaccine 19:1496-502.

45. Kirnbauer R, Chandrachud L M, O'Neil B W, Wagner E R, Grindlay G J, Armstrong A, McGarvie G M, Schiller J T, Lowy D R, Campo M S (1996) Virus-like particles of bovine papillomavirus type 4 in prophylactic and therapeutic vaccination. Virology 219:37-44

46. Knowles, G., G. J. Grindlay, M. S. Campo, L. M. Chandrachud, and B. W. O'Neil. 1997. Linear B-cell epitopes in the N-terminus of L2 of bovine papillomavirus type 4. Res Vet Sci 62:289-91.

47. Ko, K., Y. Tekoah, P. M. Rudd, D. J. Harvey, R. A. Dwek, S. Spitsin, C. A. Hanlon, C. Rupprecht, B. Dietzschold, M. Golovkin, and H. Koprowski. 2003. Function and glycosylation of plant-derived antiviral monoclonal antibody. Proc Natl Acad Sci USA 100:8013-8.

48. Koutsky L (1997) Epidemiology of genital papillomavirus infection. Am. Med. J. 102:3-8.

49. Koutsky L A, Ault K A, Wheeler C M, Brown D R, Barr E, Alvarez F B, Chiacchierini L M, Jansen K U; Proof of Principle Study Investigators (2002) A controlled trial of a human papillomavirus type 16 vaccine. N Engl J Med. 347:1645-51.

50. Kratz, P. A., B. Bottcher, and M. Nassal. 1999. Native display of complete foreign protein domains on the surface of hepatitis B virus capsids. Proc Natl Acad Sci USA 96:1915-20.

51. Leong, S. R., L. DeForge, L. Presta, T. Gonzalez, A. Fan, M. Reichert, A. Chuntharapai, K. J. Kim, D. B. Tumas, W. P. Lee, P. Gribling, B. Snedecor, H. Chen, V. Hsei, M. Schoenhoff, V. Hale, J. Deveney, I. Koumenis, Z. Shahrokh, P. McKay, W. Galan, B. Wagner, D. Narindray, C. Hebert, and G. Zapata. 2001. Adapting pharmacokinetic properties of a humanized anti-interleukin-8 antibody for therapeutic applications using site-specific pegylation. Cytokine 16:106-19.

52. Lin, Y. L., L. A. Borenstein, R. Selvakumar, R. Ahmed, and F. O. Wettstein. 1992. Effective vaccination against papilloma development by immunization with L1 or L2 structural protein of cottontail rabbit papillomavirus. Virology 187:612-9.

53. Ma, J. K., A. Hiatt, M. Hein, N. D. Vine, F. Wang, P. Stabila, C. van Dolleweerd, K. Mostov, and T. Lehner. 1995. Generation and assembly of secretory antibodies in plants. Science 268:716-9.

54. Ma, J. K., B. Y. Hikmat, K. Wycoff, N. D. Vine, D. Chargelegue, L. Yu, M. B. Hein, and T. Lehner. 1998. Characterization of a recombinant plant monoclonal secretory antibody and preventive immunotherapy in humans. Nat Med 4:601-6.

55. McCormick, A. A., M. H. Kumagai, K. Hanley, T. H. Turpen, I. Hakim, L. K. Grill, D. Tuse, S. Levy, and R. Levy. 1999. Rapid production of specific vaccines for lymphoma by expression of the tumor-derived single-chain Fv epitopes in tobacco plants. Proc Natl Acad Sci USA 96:703-8.

56. McCormick, A. A., S. J. Reinl, T. I. Cameron, F. Vojdani, M. Fronefield, R. Levy, and D. Tuse. 2003. Individualized human scFv vaccines produced in plants: humoral anti-idiotype responses in vaccinated mice confirm relevance to the tumor Ig. J Immunol Methods 278:95-104.

57. Milne, J. L., D. Shi, P. B. Rosenthal, J. S. Sunshine, G. J. Domingo, X. Wu, B. R. Brooks, R. N. Perham, R. Henderson, and S. Subramaniam. 2002. Molecular architecture and mechanism of an icosahedral pyruvate dehydrogenase complex: a multifunctional catalytic machine. Embo J 21:5587-98.

58. Padgett, H. S., A. A. Vaewhongs, F. Vojdani, and M. L. Smith. 2003. Nucleic acid molecules encoding CEL I endonuclease and methods of use thereof. United States of America patent application 20030157495.

59. Padgett, H. S., A. A. Vaewhongs, F. Vojdani, M. L. Smith, J. A. Lindbo, and W. P. Fitzmaurice. 2003. Mismatch endonucleases and methods of use. United States of America patent application.

60. Padgett, H. S., and A. A. Vaewhongs. 2003. Nucleic acid molecules encoding endonucleases and methods of use thereof. United States of America patent application 20030148315.

61. Padgett, H. S., J. A. Lindbo, and W. P. Fitzmaurice. 2002. Method of increasing complementarity in a heteroduplex. USA patent application 20020146732.

62. Padgett, H. S., J. A. Lindbo, and W. P. Fitzmaurice. 2003. Method of increasing complementarity in a heteroduplex. United States of America patent application 20030186261.

63. Padgett, H. S., W. P. Fitzmaurice, and J. A. Lindbo. 2003. Methods for homology-driven reassembly of nucleic acid sequences. United States of America patent application 20030036641.

64. Pastrana, D. V., C. B. Buck, Y. Y. Pang, C. D. Thompson, P. E. Castle, P. C. FitzGerald, S. Kruger Kjaer, D. R. Lowy, and J. T. Schiller. 2004. Reactivity of human sera in a sensitive, high-throughput pseudovirus-based papillomavirus neutralization assay for HPV16 and HPV18. Virology 321:205-16.

65. Peñaloza-Plascencia M, Montoya-Fuentes H, Florez-Martinez S E, Fierro-Velasco F J, Peñaloza-Gonzalez J M, Sánchez-Corona J (2000) Molecular identification of 7 human papillomavirus types in recurrent respiratory papillomatosis. Arch. Otolaryngol. Head Neck Surg. 126:1119-1123.

66. Pew Charitable Trusts. 2003. Pharming the Field: A look at the benefits and risks of bioengineering plants to produce pharmaceuticals. Proceedings of the conference. Pew Charitable Trusts.

67. Pogue, G. P., J. A. Lindbo, S. J. Garger, and W. P. Fitzmaurice. 2002. Making an ally from an enemy: plant virology and the new agriculture. Annu Rev Phytopathol 40:45-74.

68. Pumpens, P., and E. Grens. 2001. HBV core particles as a carrier for B cell/T cell epitopes. Intervirology 44:98-114.

69. Reddy, S. A., D. Czerwinski, R. Rajpaksa, S. J. Reinl, S. J. Garger, T. Cameron, J. Barrett, J. M. Novak, R. B. Holtz, and R. Levy. 2002. Presented at the American Society for Hematology, Orlando Fla.

70. Reddy, S. A., D. Czerwinski, S. J. Reinl, R. Rajapaksa, R. Hajnal, T. Cameron, A. A. McCormick, S. J. Garger, J. Barrett, J. M. Novak, D. Tuse, R. B. Holtz, and R. Levy. 2004. Plant derived single chain Fv idiotype vaccines in patients with follicular lymphoma: results of a phase I study, MS In Preparation.

71. Reeves W C, Ruparelia S S, Swanson K I, Derkay C S, Marcus A, Unger E R, for the RRP Taskforce. (2003) National registry for juvenile-onset recurrent respiratory papillomatosis. Archives of Otolaryngol. Head Neck Surg. 129:976-982.

72. Roden, R. B., A. Armstrong, P. Haderer, N. D. Christensen, N. L. Hubbert, D. R. Lowy, J. T. Schiller, and R. Kirnbauer. 1997. Characterization of a human papillomavirus type 16 variant-dependent neutralizing epitope. J Virol 71:6247-52.

73. Roden, R. B., E. M. Weissinger, D. W. Henderson, F. Booy, R. Kirnbauer, J. F. Mushinski, D. R. Lowy, and J. T. Schiller. 1994. Neutralization of bovine papillomavirus by antibodies to L1 and L2 capsid proteins. J Virol 68:7570-4.

74. Roden, R. B., H. L. Greenstone, R. Kirnbauer, F. P. Booy, J. Jessie, D. R. Lowy, and J. T. Schiller. 1996. In vitro generation and type-specific neutralization of a human papillomavirus type 16 virion pseudotype. J Virol 70:5875-83.

75. Rollman, E., L. Arnheim, B. Collier, D. Oberg, H. Hall, J. Klingstrom, J. Dillner, D. V. Pastrana, C. B. Buck, J. Hinkula, B. Wahren, and S. Schwartz. 2004. HPV-16 L1 genes with inactivated negative RNA elements induce potent immune responses. Virology 322:182-9.

76. Savelyeva, N., R. Munday, M. B. Spellerberg, G. P. Lomonossoff, and F. K. Stevenson. 2001. Plant viral genes in DNA idiotypic vaccines activate linked CD4+ T-cell mediated immunity against B-cell malignancies. Nat Biotechnol 19:760-4.

77. Shivprasad, S., G. P. Pogue, D. J. Lewandowski, J. Hidalgo, J. Donson, L. K. Grill, and W. O. Dawson. 1999. Heterologous sequences greatly affect foreign gene expression in tobacco mosaic virus-based vectors. Virology 255:312-23.

78. Shykhon M, Kuo M, Pearman K (2002) Recurrent respiratory papillomatosis. Clin. Otolaryngol. 27:237-243.

79. Silverberg M J, Thorsen P, Lindeberg H, Grant L A, Shah K V (2003) Condyloma in pregnancy is strongly predictive of juvenile-onset recurrent respiratory papillomatosis. Obstetrics and Gynecology 101:645-52

80. Silverberg, M. J., P. Thorsen, H. Lindeberg, L. A. Grant, and K. V. Shah. 2003. Condyloma in pregnancy is strongly predictive of juvenile-onset recurrent respiratory papillomatosis. Obstet Gynecol 101:645-52.

81. Slupetzky, K., S. Shafti-Keramat, and R. Kirnbauer. 2004. Presented at the 21st International Papillomavirus Conference, Mexico City.

82. Sokolowski, M., H. Furneaux, and S. Schwartz. 1999. The inhibitory activity of the AU-rich RNA element in the human papillomavirus type 1 late 3′ untranslated region correlates with its affinity for the elav-like HuR protein. J Virol 73:1080-91.

83. Studentsov, Y. Y., G. Y. Ho, M. A. Marks, R. Bierman, and R. D. Burk. 2003. Polymer-based enzyme-linked immunosorbent assay using human papillomavirus type 16 (HPV16) virus-like particles detects HPV16 clade-specific serologic responses. J Clin Microbiol 41:2827-34.

84. Studentsov, Y. Y., M. Schiffman, H. D. Strickler, G. Y. Ho, Y. Y. Pang, J. Schiller, R. Herrero, and R. D. Burk. 2002. Enhanced enzyme-linked immunosorbent assay for detection of antibodies to virus-like particles of human papillomavirus. J Clin Microbiol 40:1755-60.

85. Sun J D, Weatherly R A, Koopmann C F Jr, Carey T E (2000) Mucosal swabs detect HPV in laryngeal papillomatosis patients but not family members. International J. Pediatr. Otorhinolaryngol. 53:95-103.

86. Sun, X. Y., I. Frazer, M. Muller, L. Gissmann, and J. Zhou. 1995. Sequences required for the nuclear targeting and accumulation of human papillomavirus type 6B L2 protein. Virology 213:321-7.

87. Suzich, J. A., S. J. Ghim, F. J. Palmer-Hill, W. I. White, J. K. Tamura, J. A. Bell, J. A. Newsome, A. B. Jenson, and R. Schlegel. 1995. Systemic immunization with papillomavirus L1 protein completely prevents the development of viral mucosal papillomas. Proc Natl Acad Sci USA 92:11553-7.

88. Tobery T W, Smith J F, Kuklin N, Skulsky D, Ackerson C, Huang L, Chen L, Cook J C, McClements W L, Jansen K U (2003) Effect of vaccine delivery system on the induction of HPV16L1-specific humoral and cell-mediated immune responses in immunized rhesus macaques. Vaccine 21:1539-47

89. Trus, B. L., R. B. Roden, H. L. Greenstone, M. Vrhel, J. T. Schiller, and F. P. Booy. 1997. Novel structural features of bovine papillomavirus capsid revealed by a three-dimensional reconstruction to 9 A resolution. Nat Struct Biol 4:413-20.

90. White, W. I., S. D. Wilson, F. J. Palmer-Hill, R. M. Woods, S. J. Ghim, L. A. Hewitt, D. M. Goldman, S. J. Burke, A. B. Jenson, S. Koenig, and J. A. Suzich. 1999. Characterization of a major neutralizing epitope on human papillomavirus type 16 L1. J Virol 73:4882-9.

91. Yang, R., P. M. Day, W. H. t. Yutzy, K. Y. Lin, C. F. Hung, and R. B. Roden. 2003. Cell surface-binding motifs of L2 that facilitate papillomavirus infection. J Virol 77:3531-41.

What is claimed is:

1. A human papilloma virus vaccine comprising a fusion protein containing a peptide having the amino acid sequence of an epitope of HPV L2.

2. The vaccine of claim 1 wherein the peptide contains the HPV L2 epitope encoded by amino acid sequence 69-81 or 108-120.

3. The vaccine of claim 2 comprising a VLP having coat proteins of the fusion protein.

4. The vaccine of claim 1 wherein the epitope of HPV L2 is from HPV 6 or HPV 11.

5. A pediatric vaccine composition with an active ingredient of a HPV antigen in a pediatric dosage.

6. The pediatric vaccine of claim 5 wherein the HPV antigen is a fusion protein containing a peptide with the amino acid sequence of an epitope of HPV L2.

7. The pediatric vaccine of claim 6 wherein the peptide contains the epitope of HPV L2 encoded by amino acid sequence 69-81 or 108-120.

8. The pediatric vaccine of claim 7 comprising a VLP having coat proteins of the fusion protein.

9. The pediatric vaccine of claim 6 wherein the epitope of HPV L2 is from HPV 6 or HPV 11.

10. The pediatric vaccine of claim 5 in aerosol form.

11. A passive immune therapy composition comprising an protein capable of specifically binding to the neutralizing epitope of L2 of HPV 6 or HPV 11 and capable of neutralizing HPV 6 or HPV 11.


SEQUENCE LISTING (2 sequences)

SEQ ID NO: 1 - protein, 22 aa, Human papillomavirus
ASATQLYQTCKATGTCPPDVIP

SEQ ID NO: 2 - protein, 38 aa, Human papillomavirus
PPLVEPVAPSDPSIVSLIEESAIINAGAPEVVPPTQGF